(19) United States
(12) Patent Application Publication
(10) Pub. No.: US 2002/0142345 A1
(43) Pub. Date: Oct. 3, 2002

(54) Methods for encoding and decoding complex mixtures in arrayed assays

(76) Inventors: Anita J. Nelsen, Raleigh, NC (US); Lottie L. Peppers, Apex, NC (US); Michael Phillip Weiner, Cary, NC (US)

Correspondence Address: NEEDLE & ROSENBERG P C, 127 PEACHTREE STREET N E, ATLANTA, GA, 30303-1811, US

(21) Appl. No.: 09/747,003

(22) Filed: Dec. 22, 2000

(51) Int. Cl. C12Q 1/68; G01N 33/53; G06F 19/00

(52) U.S. Cl. 435/7.1; 435/6; 702/19

(57) ABSTRACT

The present invention provides a method of encoding a complex mixture of assay constituents comprising using combinations of detectable tags and a total number of detectable tags that is less than the total number of constituents to be encoded. More specifically, the method further comprises determining the total number of constituents to be encoded; determining the number of detectable tags in each combination, wherein the number of detectable tags in each combination is more than one and less than or equal to the number of prime numbers in the number of constituents to be encoded; and determining the total number of detectable tags, wherein the total number of detectable tags equals a sum of a set of factors of the total number of constituents, wherein the number of factors equals the number of detectable tags in each combination. The invention further provides using the encoding methods in a method of performing a multiplexed assay using complex mixtures of assay constituents and a kit for performing the multiplexed assay using complex mixtures of encoded assay constituents.

### BACKGROUND OF THE INVENTION

1. Field of the Invention

This invention relates generally to the fields of molecular biology and chemical analysis. More specifically, the invention relates to methods of encoding and decoding complex mixtures in multiplexed assays in order to minimize time and expense necessary for assaying numerous constituents in a single assay.

2. Background Art

In the past few years the genomes of several organisms have been completely (or nearly completely) sequenced, including those of Saccharomyces cerevisiae, Drosophila melanogaster, Escherichia coli, Caenorhabditis elegans and, most recently, the human genome. To make use of this wealth of available genomic data, rapid, high-throughput methods for analyzing all of the predicted gene products and their roles in the structural and functional organization of the cell were needed. Specifically needed were encoding and decoding means for analyzing the functional information of the thousands of genes in a complete genome.

One technology previously introduced used unique “bar-coding” tags for each of thousands of yeast genes and a silicon chip for decoding (Shoemaker et al., 1996). This method was useful for examining differential gene expression of a population of yeast strains. However, it required several thousand tags and a relatively expensive readout platform.

Prior to the present invention, no multiplexed method had been provided that could be scaled to accommodate assays of varying complexity using multiplexed, inexpensive, high-throughput methods.

### SUMMARY OF THE INVENTION

In accordance with the purpose(s) of this invention, as embodied and broadly described herein, this invention, in one aspect, relates to a method of encoding a complex mixture of assay constituents comprising using combinations of detectable tags and a total number of detectable tags that is less than the total number of constituents to be encoded. More specifically, the invention relates to a method further comprising determining the total number of constituents to be encoded; determining the number of detectable tags in each combination, wherein the number of detectable tags in each combination is more than one and less than or equal to the number of prime numbers in the number of constituents to be encoded; and determining the total number of detectable tags, wherein the total number of detectable tags equals a sum of a set of factors of the total number of constituents, wherein the number of factors equals the number of detectable tags in each combination.

In yet another aspect, the invention relates to a method of performing a multiplexed assay using complex mixtures of assay constituents encoded according to the encoding method of the invention. Specifically, the invention relates to a method comprising performing an assay to produce assay constituents using an array of the complex mixtures, wherein each constituent in a single complex mixture is detectably tagged with a unique combination of detectable tags; detecting which complex mixtures of assay constituents in the array have a positive response; and decoding the constituents in the complex mixtures having the positive response to determine which specific constituent or constituents are positive.

In another embodiment, the invention relates to a kit for performing a multiplexed assay using complex mixtures of encoded assay constituents, comprising a means of detectably tagging assay constituents encoded according to the encoding method of the invention, and an arraying means for a plurality of complex mixtures, and a container therefor.

The advantages of this invention include scalability, throughput and low-cost as compared to the currently available methods. Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and, together with the description, serve to explain the principles of the invention.

FIG. 1 shows a schematic of the bait vector, pMW101, and the prey vector, pMAR101, used in the Yeast Two Hybrid (Y2H) study.

FIG. 2 shows a schematic of the synthesis of the 96 pMAR101 prey vectors used in the Y2H study. Each 5′ ZipCode was bracketed by a similar DNA sequence (5′-TGGGCGACTTCTCCAAAC-3′, (SEQ ID NO:2) which was labeled the “Watson” sequence). And each 3′ sequence was bracketed by a second DNA sequence (5′-CTTGCAGATTCGGCAGTT-3′ (SEQ ID NO:3), which was labeled the “NCrick” sequence). PCR amplification was used to generate 96 different fragments of the Cm^{r} gene; each fragment with the following order: 5′-Watson-ZipCode₁₋₁₂-Cm^{r}-ZipCode_{A-H}-NCrick-3′. Fragments were cloned into the pMAR101 vector at a unique SwaI site.

FIG. 3 shows the method of bead-based genotyping by hybridization to Luminex beads, which was used to decode the Y2H positive wells. Following the Y2H assay, clones in positive wells were PCR amplified using biotinylated Watson and nCrick primers. For a given fragment, querying which pair of 3′ and 5′ ZipCodes were contained therein involved hybridizing the fragment to the cZipCodes on the microsphere. Flow cytometry was used to detect the label captured on a particular pair of microspheres.

FIG. 4 shows an example of a decode of 20 PCR products hybridized to a set of 20 ZipCode beads. A set of 96 vectors, each encoding a unique region containing two ZipCodes bracketed by a Watson and nCrick was used. DNA sequence served as a PCR template in a reaction containing Watson and nCrick primers. The PCR product was then used in a microsphere-based genotyping method and both of the ZipCodes on either side of the Cmr gene were decoded by hybridization to a set of 20 different beads. Shown are the MFI values obtained from the first twenty PCR products of the 96-member set.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, kits, and/or methods are disclosed and described, it is to be understood that this invention is not limited to specific assay methods, specific means or methods of detection, or to particular encoding or decoding means, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a microsphere” includes mixtures of various microspheres, reference to “an assay constituent” includes mixtures of two or more constituents, and the like.

Ranges may be expressed herein as from “about” one particular value and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint and independently of the other endpoint.

In this specification and in the claims that follow, reference will be made to a number of terms that shall be defined to have the following meanings:

“Optional” or “optionally” means that the subsequently described event or circumstance may or may not occur, and that the description includes instances where said event or circumstance occurs and instances where it does not. For example, the phrase “detectable tags optionally are contained in or coupled to microspheres” means that the detectable tags may or may not be contained in or coupled to microspheres and that the description includes both detectable tags contained in or coupled to microspheres and detectable tags otherwise used to label the desired assay constituents.

The present invention provides a method of encoding a complex mixture of assay constituents comprising using combinations of detectable tags and a total number of detectable tags that is less than the total number of constituents to be encoded. This method offers an advantage over the prior art because it reduces the number of labels necessary to detect a given number of constituents and lends itself to highly complex, multiplexed formats that are useful in high-throughput assays with pooled samples.

As used throughout, “encoding” refers to tagging an assay constituent with one or more detectable tags so that the tag(s) can be detected and the constituent identified by decoding (i.e., attributing the detectable tag(s) to a specific assay constituent).

An assay constituent can be either a reactant or a product of the assay. The constituents are selected from the group consisting of proteins, peptides, amino acids, small molecules, nucleotides, fatty acids, sugars, cofactors, receptors, receptor ligands, protein domains, oligonucleotides, transcription factors, nucleic acids, and small compounds.

As used throughout, “an assay” can be a chemical assay, protein assay, pharmacologic assay, hybrid assay (e.g., yeast two hybrid, prokaryotic two-hybrid, reverse-two hybrid, or three-hybrid assay), display assay (e.g., phage display-, F pilli- and lacI-fusion), protein readout assay, binding assay (ligand, nucleic acid, antibody, small molecule, or small compound binding assay), cell-based assay, genomic assay, read-out assay (transcriptional or protein read-out assay), or the like.

A “detectable tag” refers to any label that can be detected with a detection means and can include the absence of a label. Thus, if one hundred thousand assay constituents are to be encoded, the methods of the present invention provide that less than one hundred thousand detectable tags are used, even if one of those tags is the absence of a label. Preferably, the detectable tags are directly or indirectly coupled to the constituents. The detectable tags as used in the methods of the present invention optionally are contained in or coupled to a solid support that binds the constituents either directly or through an intermediary. Thus, the detectable tags can be coupled to a non-mobile solid support, like a plate or a chip, or a mobile solid support, like microspheres. Optionally, each detectably tagged microsphere used in the methods of the present invention is coupled to a means of specifically binding a constituent. For example, in one embodiment, the coupled means is a nucleic acid (called a “ZipCode”), which is complementary to a nucleic acid in or bound to the constituent to be encoded.

Preferably the detectable tags are selected from the group consisting of radiolabels, dyes, fluorescent labels, Quantum Dot® (Quantum Dot Corp.), and combinations thereof. “Dyes” include, but are not limited to, chemiluminescent, magnetic, and radiofrequency labels.

Optionally, the method of the present invention further comprises determining the total number of constituents to be encoded; determining the number of detectable tags in each combination, wherein the number of detectable tags in each combination is more than one and less than or equal to the number of prime numbers in the number of constituents to be encoded; and determining the total number of detectable tags, wherein the total number of detectable tags equals a sum of a set of factors of the total number of constituents, wherein the number of factors equals the number of detectable tags in each combination. For example, if 100,000 genes are to be screened using a traditional method of encoding for each assay constituent to be screened, then 100,000 different detectable tags would have to be used in a traditional one dimensional assay (i.e., one detectable tag for each constituent). In the present method, however, the number of different detectable tags can be reduced using a multi-step process. Where the total number of constituents to be screened is 100,000, the number of detectable tags in each combination can be any number between two and ten, because ten is the number of prime numbers that are multiples of 100,000 (i.e., 2×2×2×2×2×5×5×5×5×5=10 prime numbers). Thus, if three detectable tags will be used in each combination to encode a single constituent, the total number of detectable tags needed is calculated by determining three factors of the total number of constituents (e.g., 10×100×100) and adding those three factors together (i.e., 210) to determine the total number of detectable tags. Using this paradigm, the entire 100,000 genes could be screened using a total of 210 detectable tags.

TABLE 1
Examples of assay arrays and detectable tags for an assay of 100,000
constituents

| | | | Example total |
|---|---|---|---|
| | | | number of |
| Number of detectable tags in each | Example number of constituents to | | detectable tags |
| combination | be screened (factors) | | needed |
| 1 | 100,000 | (100,000) | 100,000 |
| 2 | 100,000 | (250 × 400) | 650 |
| | | (500 × 200) | 700 |
| | | (160 × 625) | 785 |
| | | (125 × 800) | 925 |
| | | (100 × 1,000) | 1,100 |
| | | (80 × 1250) | 1,330 |
| | | (40 × 2,500) | 2,540 |
| | | (50 × 2000) | 2050 |
| | | (32 × 3125) | 3,157 |
| | | (25 × 4000) | 4,025 |
| | | (20 × 5,000) | 5,020 |
| | | (16 × 6,250) | 6,266 |
| | | (10 × 10,000) | 10,010 |
| | | (8 × 12,500) | 12,508 |
| | | (5 × 20,000) | 20,005 |
| | | (4 × 25,000) | 25,004 |
| | | (2 × 50,000) | 50,002 |
| 3 | 100,000 | (50 × 25 × 80) | 155 |
| | | (100 × 25 × 40) | 165 |
| | | (32 × 25 × 125) | 182 |
| | | (20 × 125 × 40) | 185 |
| | | (16 × 125 × 50) | 191 |
| | | (10 × 100 × 100) | 210 |
| | | (160 × 25 × 25) | 210 |
| | | (10 × 125 × 80) | 215 |
| | | (8 × 125 × 100) | 233 |
| | | (20 × 25 × 200) | 245 |
| | | (160 × 5 × 125) | 290 |
| | | (16 × 25 × 250) | 291 |
| | | (100 × 5 × 200) | 305 |
| | | (50 × 8 × 250) | 308 |
| | | (4 × 125 × 200) | 329 |
| | | (250 × 5 × 80) | 335 |
| | | (250 × 4 × 100) | 354 |
| | | (10 × 25 × 400) | 435 |
| | | (250 × 2 × 200) | 452 |
| | | (50 × 5 × 400) | 455 |
| | | (400 × 2 × 125) | 527 |
| | | (8 × 25 × 500) | 533 |
| | | (500 × 5 × 40) | 545 |
| | | (50 × 4 × 500) | 554 |
| | | (100 × 2 × 500) | 602 |
| | | (8 × 625 × 20) | 653 |
| | | (16 × 625 × 10) | 651 |
| | | (32 × 5 × 625) | 662 |
| | | (4 × 625 × 40) | 669 |
| | | (2 × 625 × 80) | 707 |
| | | (800 × 5 × 25) | 830 |
| | | (10 × 10 × 1000) | 1,020 |
| | | (20 × 5 × 1000) | 1,025 |
| | | (4 × 25 × 1000) | 1,029 |
| | | (50 × 2 × 1000) | 1,052 |
| | | (10 × 8 × 1250) | 1,268 |
| | | (16 × 5 × 1250) | 1,271 |
| | | (1250 × 4 × 20) | 1,274 |
| | | (1250 × 2 × 40) | 1,292 |
| | | (10 × 5 × 2000) | 2,015 |
| | | (2 × 25 × 2000) | 2,027 |
| | | (8 × 5 × 2500) | 2,513 |
| | | (10 × 4 × 2500) | 2,514 |
| | | (20 × 2 × 2500) | 2,522 |
| | | (4 × 3125 × 8) | 3,137 |
| | | (2 × 3125 × 16) | 3,143 |
| | | (4,000 × 5 × 5) | 4,010 |
| | | (2 × 125 × 4000) | 4,127 |
| | | (10 × 2 × 5000) | 5,012 |
| | | (4 × 5 × 5000) | 5,009 |
| | | (6,250 × 4 × 4) | 6,258 |
| | | (6,250 × 2 × 8) | 6,260 |
| | | (2 × 5 × 10,000) | 10,007 |
| | | (12,500 × 2 × 4) | 12,506 |
| | | (25,000 × 2 × 2) | 25,004 |
| 4 | 100,000 | (e.g., 10 × 10 × 10 × 100) | 130 |
| 5 | 100,000 | (e.g., 10 × 10 × 10 × 10 × 10) | 50 |
| 6 | 100,000 | (e.g., 2 × 5 × 10 × 10 × 10 × 10) | 47 |
| 7 | 100,000 | (e.g., 2 × 2 × 5 × 5 × 10 × 10 × 10) | 44 |
| 8 | 100,000 | (e.g., 2 × 2 × 2 × 5 × 5 × 5 × 10 × 10) | 41 |
| 9 | 100,000 | (e.g., 2 × 2 × 2 × 2 × 5 × 5 × 5 × 5 × 10) | 38 |
| 10 | 100,000 | (2 × 2 × 2 × 2 × 2 × 5 × 5 × 5 × 5 × 5) | 35 |

As demonstrated by the above table, the total number of detectable tags is minimized by selecting factors of the total number of constituents that are equal or approximate. Thus, in one embodiment of the present method, the total number of detectable tags is minimized by selecting factors of the total number of constituents that are equal or approximate.

Using the encoding method of the present invention, an assay can be designed based on the total number of detectable tags available, based on the total number of tags in each combination, based on the arraying means (e.g., the number of wells on a plate that can be read using automated readers), or a combination. Thus, if 100,000 constituents are to be assayed and only 35 total detectable tags are available, then there must be 10 detectable tags in each combination. Alternatively, if there are practical limitations to the number of tags that can be detected in combination, then the assay could limit the number of detectable tags in combination to, for example, three and the total number of detectable tags could be, for example, 210.

The invention further provides a method of performing a multiplexed assay using complex mixtures of assay constituents encoded according to the encoding method of the invention. Specifically, the method comprises performing an assay to produce assay constituents using an array of the complex mixtures, wherein each constituent in a single complex mixture is detectably tagged with a unique combination of detectable tags; detecting which complex mixtures of assay constituents in the array have a positive response; and decoding the constituents in the complex mixtures having the positive response to determine which specific constituent or constituents are positive. As used herein, “an array” includes a multiwell plate or any other arraying means. Thus, an array using a multiwell plate can be eight wells in one dimension and twelve wells in another dimension as in a 96 well plate. An array could also be sixteen wells in one dimension and twelve wells in another dimension, using two 96 well plates.

The detection means is selected as specific for the detectable tags. For example, if the detectable tag is fluorescent and is contained in or coupled to microspheres, then flow cytometry with a fluorescence detection device or a FAC sorter can be used to detect and distinguish a tag or combination of tags. When a specific mixture (e.g., a complex mixture in a specific well in an assay plate) has a positive response, then that particular mixture is decoded to identify the positive constituent in that mixture. For example, if the well contained ten genes to be screened, then the decoding method would identify which of the ten genes had a positive response. Thus, the decoding is performed by detecting and distinguishing with a detection device the detectable tags in each complex mixture of constituents.

In one embodiment the method of encoding and decoding is used with a complex mixture of arrayed cDNA clones in a yeast two-hybrid analysis. The steps comprise using an array of complex mixtures of yeast host cells comprising an encoded set of cDNAs made by cloning each individual cDNA into a member of a set of vectors, wherein each member of the set of vectors comprises a yeast two-hybrid activation domain and a selected pair of identifying nucleic acid sequences (“ZipCodes”), wherein the selected pair of identifying nucleic acids is specific for each individual cDNA, and wherein the yeast host cells containing the set of vector are combined to create complex mixtures of cDNA clones; mating the arrayed host cells with a yeast expressing a bait protein and one or more reporter genes; detecting an interaction or absence of an interaction between the bait protein and the activation domains in each complex mixture of the array by determining the expression of the reporter gene or genes; performing PCR amplification of each complex mixture that shows an interaction, wherein the PCR amplification is performed using labeled primers; and decoding the PCR products using a genotyping assay. In one embodiment, the first member of the selected pair of identifying nucleic acids is at the 5′ end of an antibiotic resistance gene and the second member is at the 3′ end of the antibiotic resistance gene. Preferably, each vector in the set has two primer nucleic acid sequences present in each vector, wherein the first primer nucleic acid is at the 5′ end of the first member of the identifying pair and the second primer nucleic acid is at the 3′ end of the second member of the identifying pair. In one embodiment, the antibiotic resistance gene is a chloramphenicol gene.

In one embodiment of the yeast two hybrid method, each identifying nucleic acid is 25 bases. In another embodiment, twenty different identifying nucleic acids are used in combinations to form 96 different pairs of identifying nucleic acids. Thus, each complex mixture can contain up to about 96 different cDNAs and the array of complex mixtures of cDNA clones in host cells can contain up to about 9,220 different cDNAs.

In one embodiment of the yeast two hybrid method, the reporter gene is a β-galactosidase gene. In another embodiment the reporter gene is a Leu2 gene. In yet another embodiment, the reporter genes are both the β-galactosidase gene and the Leu2 gene.

The genotyping assay as used in the yeast two hybrid method comprises contacting, under conditions that allow formation of hybridization products, the labeled PCR products of each mixture with a set of microspheres, wherein each member of the set of microspheres is distinguishably labeled and is coupled with a capture nucleic acid complementary to one of the identifying nucleic acid sequences; detecting the label of the PCR product and the label of the microsphere in two or more hybridization products. The presence of a labeled PCR product in two different hybridization products indicates the cDNA specific to the pair of identifying nucleic acid sequences. Preferably, the distinguishable label of the microsphere is a fluorescent label, wherein the PCR product is fluorescently labeled, and wherein the fluorescent label of the microsphere and the PCR product can be detected in the same reaction product or products.

In one embodiment of the yeast two hybrid method, the microspheres are carboxylated and amino groups at the 5′ end of the capture nucleic acids are coupled to the carboxyl groups.

Preferably, the capture nucleic acid further comprises a luciferase cDNA. Optionally, the luciferase cDNA has the sequence CAGGCCAAGTAACTTCTTCG (SEQ ID NO:1). The capture oligonucleotide can be directly coupled to the microsphere or can be indirectly coupled to the microsphere by a carbon spacer.

In the yeast two hybrid method, the label of the PCR product and the label of the microsphere in two or more hybridization products is preferably detected using flow cytometry.

The present invention further provides a kit for performing a multiplexed assay using complex mixtures of encoded assay constituents, comprising a means of detectably tagging assay constituents encoded according to the encoding method of the invention, and an arraying means for a plurality of complex mixtures, and a container therefor. The means of detectably tagging assay constituents can include, for example, a set of microspheres, wherein each member of the set of microspheres is detectably tagged and binds selectively to an assay constituent. For example, the kit can comprise a set of detectably tagged microspheres that bind selectively to cDNA clones in a yeast two-hybrid analysis. The kit for performing a yeast two hybrid can further comprise one or more of the following: a set of yeast vectors comprising a reporter gene, a yeast two-hybrid activation domain, and a selected pair of identifying nucleic acid sequences, wherein the selected pair of identifying nucleic acids is specific for each vector; a means for homologously recombining the cDNAs to be encoded into the vectors of the set into yeast host cells; a means for combining the yeast host cells containing the set of vectors to create the complex mixture of cDNA clones; a set of yeast bait cells expressing a bait protein; a means for mating the yeast cells containing the set of vectors and the yeast bait cells; a set of labeled PCR primers; or a set of microspheres, wherein each member of the set of microspheres is detectably labeled and is coupled with a capture nucleic acid complementary to one of the identifying nucleic acid sequences.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some deviations should be accounted for. Unless indicated otherwise, parts are parts by weight, temperature is in ° C. or is at ambient temperature, and pressure is at or near atmospheric.

### Example 1

### Yeast 2-Hybrid Analysis

Reagents. Restriction and DNA modification enzymes were purchased from various manufacturers and used according to their recommendations. AmpliTaq Gold DNA polymerase and Big Dye Terminator Cycle Sequencing reagent were purchased from Applied Biosystems (Foster City, Calif., USA). Unmodified oligonucleotides were purchased from either Keystone Biosource (Camarillo, Calif., USA), MWG Research (High Point, N.C., USA) or IDT (Coralville, Iowa, USA). 2-[N-Morpholino]ethanesulfonic acid (MES) and 1-Ethyl-3-(3-Dimethylaminopropyl) carbodiimide Hydrochloride (EDC) were purchased from Sigma (St. Louis, Ill., USA) and Pierce (Rockford, Ill., USA), respectively. Streptavidin phycoerythrin was purchased from Becton Dickinson (San Jose, Calif., USA). Yeast cell preparation and transformation reagents were purchased from Zymo Research (Orange, Calif., USA).

Preparation of microspheres. Carboxylated fluorescent polystyrene microspheres (5.5 μm in diameter) were purchased from the Luminex Corp. (Austin, Tex., USA). Oligonucleotides, synthesized to contain a 5′ amino group, C₍₁₅₋₁₈₎ spacer, 20 base luciferase sequence and 25 base Zipcode-complimentary sequence, were ordered from Oligos, Etc. (Wilsonville, Oreg., USA) or from Applied Biosystems. Oligonucleotides were covalently coupled to the microspheres as described by Ianonne and co-workers (Chen et al. (2000); Iannone et al (2000)).

Yeast strains, plasmids, and media. Yeast strains EGY48 and L40 have been described (Finley & Brent (1994)). Plasmids pHybLex/Zeo, pYesTrp2 and pMW101 have also been described (Finley & Brent (1994); Gyuris et al (1993); Watson et al (1996)). Selective yeast media were prepared as described in Gyuris et al. (1993). The plasmid pBC SK+ was purchased from Stratagene (La Jolla, Calif., USA).

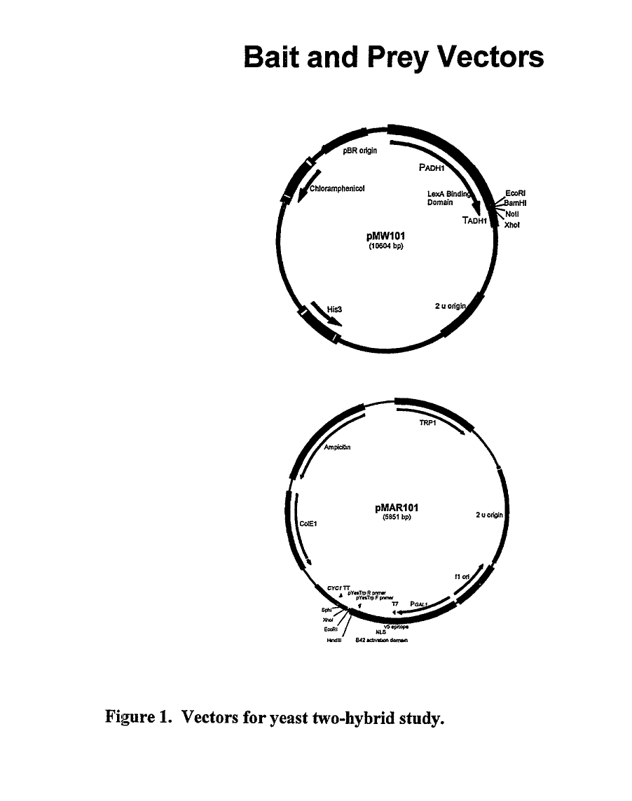

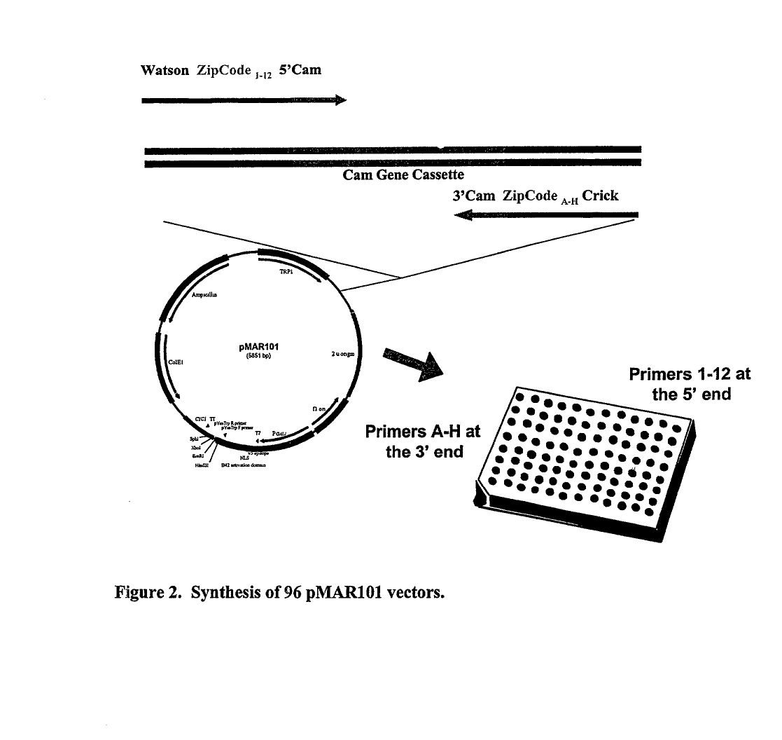

Construction of pMAR101 and pMAR101 derivatives. To construct pMAR101, a PCR product derived from the amplification of pMW101 with forward primer (5′-GCCGAAGCTTGCGGTTGGGGTATTCGCAACGGCGACTGG-3′) (SEQ ID NO:28) and reverse primer (5′-ATACGCATGCAATTCGCCCGGAATTAGCTT GGCTGCAGGT-3′) (SEQ ID NO:29) was digested with restriction endonucleases HindIII and SphI and ligated overnight at 16° C. with HindIII, SphI-digested, agarose gel purified plasmid pYesTrp2 (Invitrogen, Carlsbad, Calif.). Addition of this PCR product incorporates regions of approximately 23 bases adjacent to and on either side of the multiple cloning site. The resulting plasmid enables simultaneous homologous recombination of amplified genes into both bait and prey vectors (FIG. 1). To construct pMAR101.1-pMAR101.96 twenty primers (Table 2) containing a common sequence for amplification, a 25 base zipcode and a terminal end of the chloramphenicol (Cm^{r}) gene were used to amplify the Cm^{r} marker from the plasmid pBC SK+. Following amplification under standard conditions in Optiprime buffer #5 (Stratagene), 2 units of Pfu polymerase were added to each of the 50 μl reactions and incubated at 72° C. for 20 minutes. The resulting 96 unique blunt-ended fragments were ligated with SwaI digested pMAR101 for 16 hours at 16° C. Ligation products were transformed into electrocompetent DH10B cells (LTI, Gaitherburg, Md., USA) and clones were selected on LB agar plates containing carbenicillin (50 μg/ml) and chloramphenicol (12.5 μg/ml) (FIG. 2). Colonies were screened by PCR to confirm incorporation of the cassette as well as to select for uniform orientation of the cassette. Common sequence primers, “Watson” (5′-TGGGCGACTTCTCCAAAC-3′) (SEQ ID NO:2) and “nCrick” (5′-CTTGCAGATTCGGCAGTT-3′) (SEQ ID NO:3), were used to confirm that the 1241 bp fragment was incorporated into the plasmid. Primers Watson and a plasmid-specific oligo, “pYesTrp Forward” (Invitrogen) were used to screen for the orientation of the Cm^{r} gene.

TABLE 2
DNA Primers and Associated ZipCode Sequences
DNA Sequence^{a}

| | | | |
|---|---|---|---|
| Primer | Watson/NCrick | ZipCode | Cam gene |

| | | |
|---|---|---|
| 1 | TGGGCGACTTCTCCAAACGATGATCGACGAGACACTCTCGCCACTGTGACGGAAGATCACTTCGC | (SEQ ID NO: 4) |
| 2 | TGGGCGACTTCTCCAAACCGGTCGACGAGCTGCCGCGCAAGATCTGTGACGGAAGATCACTTCGC | (SEQ ID NO: 5) |
| 3 | TGGGCGACTTCTCCAAACGACATTCGCGATCGCCGCCCGCTTTCTGTGACGGAAGATCACTTCGC | (SEQ ID NO: 6) |
| 4 | TGGGCGACTTCTCCAAACCGGTATCGCGACCGCATCCCAATCTCTGTGACGGAAGATCACTCGC | (SEQ ID NO: 7) |
| 5 | TGGGCGACTTCTCCAAACGCTCGAAGAGGCGCTACAGATCCTCCTGTGACGGAAGATCACTTCGC | (SEQ ID NO. 8) |
| 6 | TGGGCGACTTCTCCAAACCACCGCCAGCTCGGCTTCGAGTTCGCTGTGACGGAAGATCACTTCGC | (SEQ ID NO: 9) |
| 7 | TGGGCGACTTCTCCAAACGTAAATCTCCAGCGGAAGGGTACGGCTGTGACGGAAGATCACTTCGC | (SEQ ID NO: 10) |
| 8 | TGGGCGACTTCTCCAAACCTTTTCCCGTCCGTCATCGCTCAAGCTGTGACGGAAGATCACTTCGC | (SEQ ID NO: 11) |
| 9 | TGGGCGACTTCTCCAAACGGCTGGGTCTACAGATCCCCAACTTCTGTGACGGAAGATCACTTCGC | (SEQ ID NO: 12) |
| 10 | TGGGCGACTTCTCCAAACGAACCTTTCGCTTCACCGGCCGATCCTGTGACGGAAGATCACTTCGC | (SEQ ID NO: 13) |
| 11 | TGGGCGACTTCTCCAAACTTTCGGCACGCGCGGGATCACCATCCTGTGACGGAAGATCACTTCGC | (SEQ ID NO: 14) |
| 12 | TGGGCGACTTCTCCAAACCTCGGTGGTGCTGACGGTGCAATCCCTGTGACGGAAGATCACTTCGC | (SEQ ID NO: 15) |
| A | CTTGCAGATTCGGCAGTTTCAACGTGCCAGCGCCGTCCTGGGACTCCACGGGGAGAGCCTGAGCA | (SEQ ID NO: 16) |
| B | CTTGCAGATTCGGCAGTTGCGAAGGAACTCGACGTGGACGCCGCTCCACGGGGAGAGCCTGAGCA | (SEQ ID NO: 17) |
| C | CTTGCAGATTCGGCAGTTCGGGGATACCGATCTCGGGCGCACACTCCACGGGGAGAGCCTGAGCA | (SEQ ID NO: 18) |
| D | CTTGCAGATTCGGCAGTTGGAGCTTACGCCATCACGATGCGATCTCCACGGGGAGAGCCTGAGCA | (SEQ ID NO: 19) |
| E | CTTGCAGATTCGGCAGTTCGTGGCGGTGCGGAGTTTCCCCGAACTCCACGGGGAGAGCCTGAGCA | (SEQ ID NO: 20) |
| F | CTTGCAGATTCGGCAGTTCGATCCAACGCACTGGCCAAACCTACTCCACGGGGAGAGCCTGAGCA | (SEQ ID NO: 21) |
| G | CTTGCAGATTCGGCAGTTCTGAATCCTCCAACCGGGTTGTCGACTCCACGGGGAGAGCCTGAGCA | (SEQ ID NO: 22) |
| H | CTTGCAGATTCGGCAGTTTTCGGCGCTGGCGTAAAGCTTTTGGCTCCACGGGGAGAGCCTGAGCA | (SEQ ID NO: 23) |

^{a}DNA Sequences are as follows: 3′ cam gene, TCCACGGGGAGAGCCTGAGCA (SEQ ID NO: 24); 5′ cam gene, CTGTGACGGAAGATCACTTCGC (SEQ ID NO: 25); Watson, TGGGCGACTTCTCCAAAC (SEQ ID NO: 2); NCrick, CTTGCAGATTCGGCAGTT (SEQ ID NO: 3). The ZipCode sequences (in bold) are shown between the Watson/NCrick and Cam sequences.

Preparation of pMAR101.1-pMAR101.96. pMAR101.x plasmid DNAs were purified using Qiatip-500 columns (Qiagen, Valencia, Calif., USA). The resulting DNAs were digested with EcoRI and XhoI restriction enzymes and purified on 1% preparative agarose gels. Digested plasmid was transformed into competent EGY48 cells and plated on agar plates containing YNB−Trp+glucose to determine background.

Cloning by Homologous Recombination. Plasmids were constructed in vivo in yeast as described by Oldenburg et al. (1997). Briefly, genes of interest were amplified from plasmid DNAs isolated from commercially available cDNA libraries. Primers for amplification were designed to include portions of both the pMAR101 plasmid as well as portions of the gene of interest. The forward primer contained 23 bases of vector sequence immediately adjacent to and 5′ of the EcoRI restriction site (GCAACGGCGACTGGCTGGAATTC) (SEQ ID NO:26) fused to approximately 25 bases of the 5′ end of the gene to be amplified. This primer does not require a start codon, but does require the gene to be in-frame with the EcoRI site. The reverse primer contained 23 bases of vector sequence adjacent to and 3′ of the XhoI site (GCTTGGCTGCAGGTCGACTCGAG) (SEQ ID NO:27) fused to approximately 25 bases of the 3′ end of the gene to be amplified. The 3′ primer does require a termination codon. Amplification was carried out in 25 μl reactions, each containing 100 ng cDNA template, 200 nm primers, 1× RedTaq buffer (Sigma), 200 μM dNTP mix (Sigma), 0.75 units RedTaq polymerase (Sigma) and 0.125 units Pfu polymerase. The MgCl₂ concentration was adjusted to a final concentration of 3.0 μM. Reactions were amplified for 30 cycles (94° C. for 30 seconds, 56° C. for 45 seconds and 72° C. for 2 minutes) followed by a final extension of 72° C. for 7 minutes. Products were verified by electrophoresis of 5 μl on analytical agarose gels. PCR products were then cloned into pMAR101.x by homologous recombination and co-transformed into yeast. Competent EGY48 yeast cells (10 ) were combined with 50 ng of EcoRI, XhoI digested vector, 100 ng PCR products and 100 μl EZ3 solution, vortex mixed, and incubated at 30° C. After at least 30 minutes, the entire reaction was plated on agar plates containing YNB−Trp+glucose and incubated at 30° C. for 72 hours. Colonies were screened for inserts by PCR with the vector-specific primers pYesTrp Forward and Reverse (Invitrogen). Ninety-six different genes were cloned into the 96 unique pMAR101.x vectors and pooled for analysis against the bait clones. Using a similar method, baits for Y2H were cloned and co-transformed into either pMW101/RFY206/pSH1834T or pHybLex/L40. Bait clones were selected on YNB+Ura+His+glucose or YPD+Zeocin(300 μg/ml), respectively.

Preparation of yeast bait and prey cultures. For validation experiments, several colonies from the bait transformation plates were inoculated into 15 mL of selection media and grown 48 hours at 30° C. For high-throughput experiments, 96 different baits were arrayed in 96-well V-bottom microplates containing 200 μl of selection media and grown 48 hours at 30° C. For both applications, yeast library plates (containing genes cloned into the 96 pMAR101.x vectors) were thawed and 200 μl of selection media (YNB−Trp+glucose with antibiotics) was added to each well. These plates were also incubated at 30° C. for 48 hours.

Yeast two-hybrid assay. Liquid mating of yeast was performed essentially as described in Buckholz et al. (1999). To validate this method, 5 μl from each well of the prey yeast library was transferred into a fresh 96-well V-bottom plate. Bait cultures were spun down and the cells resuspended in 45 mL YEP galactose+raffinose broth with antibiotics. A 25 μl aliquot of bait culture was added to the 5 μl of prey culture in each well. Following a 48 hour incubation at 30° C., 200 μl of minimal selective dropout media minus uracil, histidine, tryptophan and leucine, plus 2% galactose and 1% raffinose (SGR-UHWL) was added diluting the rich YPD media 1:10. After incubating an additional 48 hours, 5 μl of samples were transferred to a new microtiter plate and diluted 1:40 using SGR-UHWL to a final volume of 205 μl. The diluted matings were incubated for an additional 3-5 days at 30° C. The mating mixture (25 μl) was transferred to 96-well assay plates and β-galactosidase assay was performed as described.

For high-throughput analysis 175 μl of each prey culture was transferred to a 50 ml conical centrifuge tube and spun down, the supernatant removed, and the cells resuspended in 45 mL YNB−Trp+glucose with antibiotics. A 5 μl aliquot of each bait culture was transferred into a fresh 96-well V-bottom plate and 25 μl of the pooled prey culture was added to each well.

Decoding assay. Positive-interactors detected via the β-gal assay served as template for PCR in which the chloramphenicol cassettes of the interacting prey (pMAR101.x) were amplified using biotinylated primers, Watson and nCrick, and standard PCR conditions. Products were hybridized against a pool of microspheres containing 250 beads per μl in 1.5 M NaCl. The pool was populated with 20 different types of microspheres; where each type was coupled to the complement of one of the ZipCode sequences used in pMAR101.x construction. During the hybridization, samples were denatured at 96° C. for 2 minutes and incubated at 45° C. for greater than one hour. Following hybridization, samples were washed in 1× SSC containing 0.2% Tween-20, resuspended in a 1.5M NaCl solution containing streptavidin-phycoerythrin and incubated for 20 minutes at room temperature. The reactions were diluted with 60 μl of 1× SSC containing 0.2% Tween-20 prior to analysis on the LX100 (Luminex, Austin, Tex.).

Validating the vectors. We synthesized 96 “library” vectors, each vector identifiable by a unique pair of 2 of 20 possible 25-base ZipCodes. The ZipCodes we used were DNA sequences derived from a sequence of the M. tuberculosis genome. Chen et al. (2000); Iannone (2000). The particular ZipCode sequences were chosen to: 1) be absent from the known human genome sequence (by BLAST analysis), 2) have no discernable secondary structure, and 3) not hybridize to any of the other ZipCodes under the conditions (determined empirically) used for the genotyping and decoding analysis.

Twelve of the 20 ZipCodes (ZipCodes₁₋₁₂) have been placed 5′ upstream of a Cm^{r} gene while the other 8 (ZipCodes_{A-H}) have been located at the 3′ end of the Cm^{r} gene. Each 5′ ZipCode was bracketed by a similar DNA sequence (5′-TGGGCGACTTCTCCAAAC-3′ (SEQ ID NO:2), a translation of the amino acid sequence WATSON which we have labeled the “Watson” sequence); each 3′ sequence was bracketed by a second DNA sequence (5′-CTTGCAGATTCGGCAGTT-3′ (SEQ ID NO:3), a translation of the amino acid sequence NCRICK which we have labeled the “nCrick” sequence). PCR amplification was used to generate 96 different cassettes containing the Cm^{r} gene; each fragment with the following order: 5′-Watson-ZipCode₍₁₋₁₂₎-Cm^{r}-ZipCode_{(A-H)}-nCrick-3′. The 96 different cassettes were cloned into a unique Swa I site of the pMAR101 vector to synthesize the vectors pMAR101.1 . . . pMAR101.96.

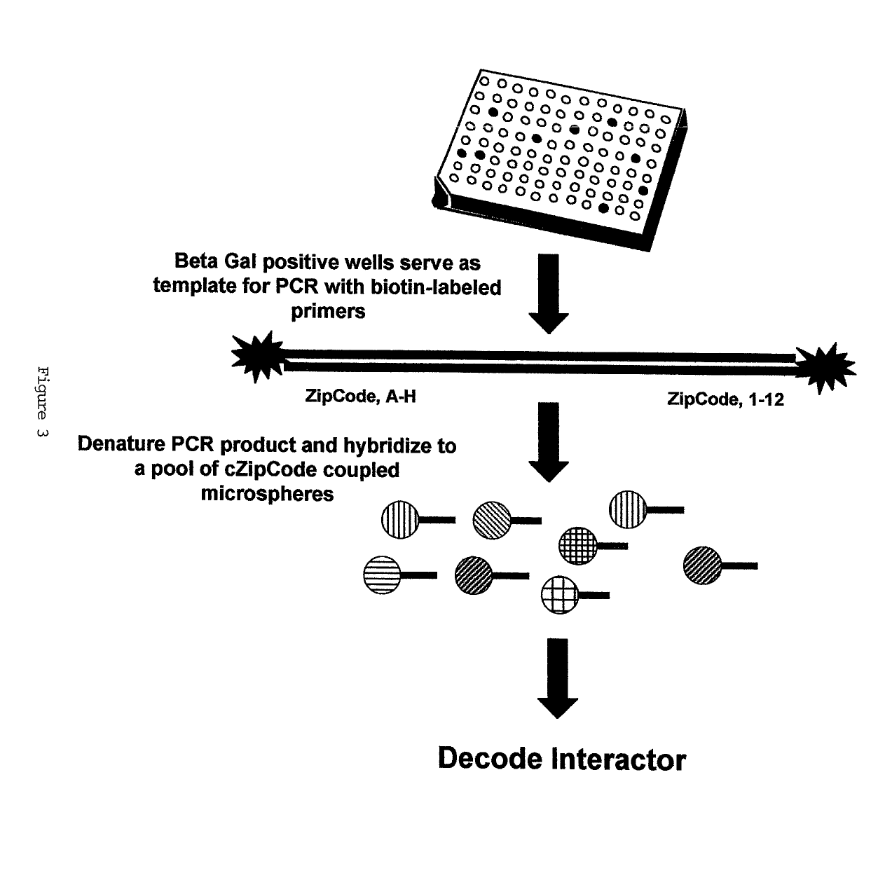

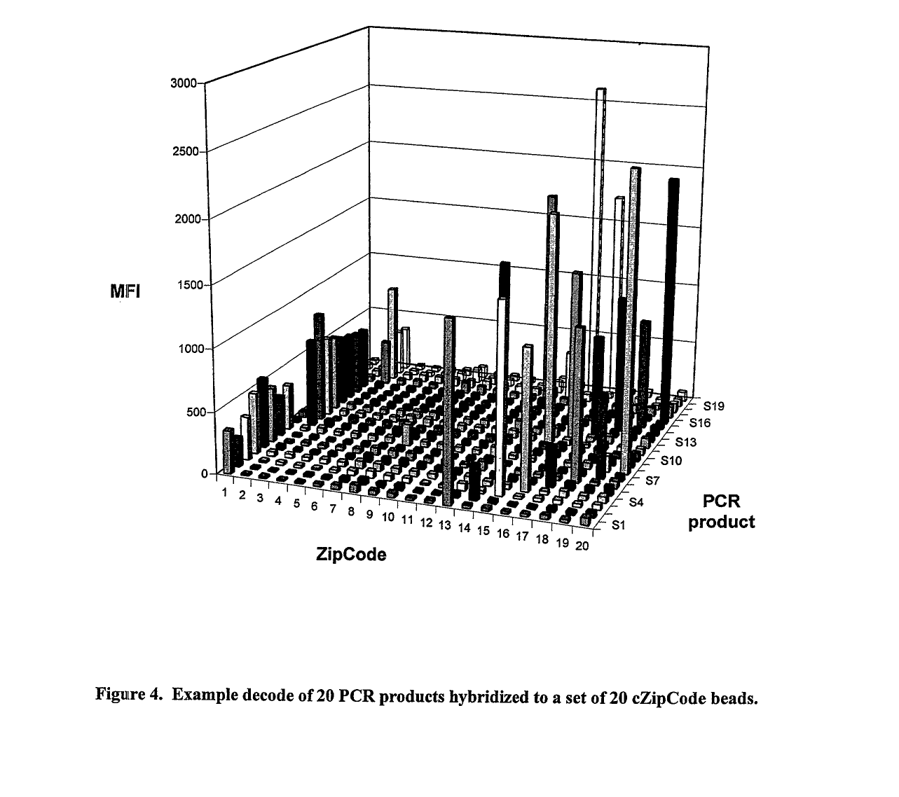

The set of vectors was purified and biotin-labeled Watson and nCrick primers were used to generate a PCR fragment. This PCR fragment was then hybridized against the set of 20 microspheres (FIG. 3). FIG. 4 illustrates the results from a subset of the analysis and demonstrates clear discrimination of the appropriate pair of the beads to the labeled fragment.

Identifying interactors using a non-random array. Prey clones were constructed in groups of (for this example) 96 where each novel cDNA fragment of the group was cloned into a unique vector of the 96 library vectors. A Y2H analysis was performed using a bait protein against the multiplexed prey. Positive clones were isolated and the cassettes containing the Cm^{r} genes were amplified using biotin-dye-labeled ‘Watson’ and ‘nCrick’ primers. The PCR product was then used in a bead-based decoding assay and both of the ZipCodes on either side of the Cm^{r} gene were identified by hybridization to a set of 20 different beads. The data points were used to decode which member of the 96 vector set contained the interactor cDNA fragment which interacted with our bait protein. To further validate this result, we used vector specific primers to amplify the region containing the cloned cDNA fragment and submitted this fragment for DNA sequencing. Results were analyzed by BLAST and compared with the results of the bead-based assay, shown in Table 3.

The data show that this method can be used for high-throughput yeast two-hybrid analysis where the proteins of interest are encoded by known or predicted cDNA sequences. The method is easily automated and can be scaled to accommodate projects of varying complexity. In this analysis of 96 clones per well, 20 ZipCodes in two “dimensions,” the dimensions containing 12 and 8 elements (i.e., a 96 well plate), respectively, were used. In practice, a reduced set of 13 elements in 6 dimensions (2×2×2×2×2×3) can be used to encode the 96 different beads. In fact, the complexity of any number of analyses per well can be reduced to the sum of the prime factors defining the maximum number of clones one desires to analyze. For example, a 10-fold increase in well complexity, i.e., 960 clones per well, (12×8×10) could be encoded by either 30 beads or by the sum of the prime factors making up 960 (2×2×2×2×2×3×2×5), i.e., with just 20 beads.

To access the complete set of human genes with just a set of the currently existing 100 bead set, it is reasonable to expect that a multiplex of 1000 clones per well could be decoded using a 10×12×8 matrix or array. A single master library plate could be used to represent close to the predicted number of genes.

TABLE 3
Example of results from an assay of approximately 1500 bait clones
against 350 prey clones.¹

| | Avg. | | | | | | |
|---|---|---|---|---|---|---|---|
| | β-gal | Bead Set Values³ | | | | Hit Identity | Sequence |
| Sample² | Score | 1 | 2 | 3 | 4 | Plasmid | Verification⁴ |
| B707.P677.F05 | 8603 | 79 C | 58 10 | 71 11 | 8 E | C10 | (+) |
| | | 2964.922 | 2155.084 | 67.3227 | 52.8565 | pMAR101.75 | |
| B707.P675.E05 | 2470 | 58 10 | 77 B | 53 H | 8 E | B10 | (+) |
| | | 1811.079 | 703.7803 | 16.65197 | 14.02155 | pMAR101.74 | |
| B691.P679.G06 | 2224 | 56 9 | 8 E | 34 2 | 81 D | E09 | (+) |
| | | 1436.165 | 761.8906 | 1.283019 | −2.06527 | pMAR101.69 | |
| B674.P675.G06 | 6480 | 52 6 | 53 H | 50 5 | 33 G | H06 | (+) |
| | | 2102.083 | 1564.84 | 18.56156 | 16.50951 | pMAR101.48 | |
| B674P675.A11 | 4556 | 38 4 | 75 A | 79 C | 53 H | A04 | (−)⁵ |
| | | 1008.487 | 351.1794 | 36.33744 | 34.78622 | pMAR101.25 | |
| B678P679.D01 | 2784 | 50 5 | 53 H | 73 12 | 52 6 | D01 | (+) |
| | | 2203.989 | 1553.246 | 36.82699 | 26.41147 | pMAR101.04 | |
| B680P679.H04 | 5444 | 36 3 | 53 H | 33 G | 50 5 | H03 | (+) |
| | | 1957.287 | 1955.586 | 333.8939 | 236.088 | pMAR101.24 | |
| B674P675.G05 | 12924 | 50 5 | 33 G | 73 12 | 79 C | G05 | (+) |
| | | 2120.277 | 1602.547 | 20.97098 | 16.38077 | pMAR101.39 | |
| B674P675.G06 | 6480 | 52 6 | 53 H | 33 G | 50 5 | H06 | (+) |
| | | 2019.826 | 1536.088 | 42.05269 | 26.03052 | pMAR101.47 | |

¹All samples have been assayed in duplicate. Samples in which both assays were successful have been included in the data.
²Sample names reflect (“bait” plate. “prey” pool. “bait” well)
³Luminex bead values for 4 highest scoring beads (out of 20-bead set) after background subtraction.
⁴Sequence results of inserts cloned by homologous recombination into pMAR101.x were analyzed by BLAST.
⁵Top BLAST hit reveals a protein family member closely related to cloned gene.

### Example 2

### Phage display-, F pilli- and lacI-fusion Display Systems

Using the protocol set forth in Example 1, 96 or more differentially-labeled M13 clones are made. These are used in either gpIII or gpVIII vectors in an arrayed format as in the Y2H example. Alternatively, the ZipCodes are generated in a ‘wild-card’ (i.e., random) synthesis and then cloned into the vectors as pools of up to several thousands to millions. These libraries are used in a typical phage display experiment and the ZipCodes identified after panning.

LacI and F pilli are other display-systems and can be used in a manner identical to the phage display methods.

### Example 3

### Baculovirus (in Insect and Eukaryotic) Adenovirus, Adeno-associated Virus (AAV), and Retrovirus

Similar to that described for phage display and yeast two-hybrid, these eukaryotic viruses are used to express foreign protein in eukaryotic cells. Baculovirus can be used to infect both insect and mammalian cells and also as a fusion vector (in a manner similar to the M13 gp-fusion vectors).

### Example 4

### Cell-based Assay, Cell-surface Marker Hapten or Cell-surface Protein

The transcriptional readout used in the Y2H system is engineered such that the reporters are either small haptens or peptides that are transcribed and that eventually appear on the surface of the cell as in vivo fusions (for example, in yeast the Mat alpha gene product, in E. coli the F pilli gene product, and in mammalian cell lines the CD40 gene product). These cell lines are used in panning experiments against a set of antibodies or other specific-interactors. The interactors are fused to beads or labeled with a reporter molecule. In a 2-dimensional analysis of 20 different fusions, 96 (using 8×12) different types of cells are analyzed at once. In a manner similar to that described for yeast two-hybrid, the number of dimensions or factors within a dimension are increased to increase the number of cell lines that could be simultaneously examined.

### Example 5

### Cell-based Assay, Other Markers

In place of haptens as in the previous example, the reporters are external protein fusions or nucleic acid molecules (for example, DNA and/or RNA). The reporter molecules can be one of the many forms of green flourescent proteins (GFPs) available.

### Example 6

### Protein Tags with Antibodies

Proteins are synthesized with genetic fusion tags. In a specific example, a set of twenty tags is designed that do not cross-react. Sets of tags are used in a manner similar to the dimensions used in the yeast two-hybrid experiment described.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Buckholz, R. G., et al. (1999) Automation of yeast two-hybrid screening. J. Molec. Microbiol. Biotechnol. 1: 34-38.

2. Chen, J., et al. (2000) A Microsphere-Based Assay for Multiplexed Single Nucleotide Plymorphism Analysis Using Single Base Chain Extension. Genome Research 10:549-557.

3. Finley, R. L., Brent, R. (1994) Interaction mating reveals binary and ternary connections between Drosophila cell cycle regulators. Proc. Natl. Acad. Sci. USA 91: 12980-12984.

4. Gyuris, J., et al. (1993) Cdi1, a human G1 and S phase protein phosphatase that associates with Cdk2. Cell 75: 791-803.

5. Iannone, M. A., et al. (2000) Multiplexed Single Nucleotide Polymorphism Genotyping by Oligonucleotide Ligation and Flow Cytometry. Cytometry 39:131-140.

6. Oldenburg, K., et al. (1997) Recombination-mediated PCR-directed plasmid construction in vivo in yeast. Nucleic Acids Research 25:451-452.

7. Shoemaker, D. D., et al. (1996) Quantitative phenotypic analysis of yeast deletion mutants using a highly parallel molecular bar-coding strategy. Nat. Genet. 14:450-456.

8. Watson, M. A., et al. (1996) Vectors encoding Alternative Antibiotic Resistance for Use in the Yeast Two-Hybrid System. BioTechniques 21:255-259.

What is claimed is:

1. A method of encoding a complex mixture of assay constituents comprising using combinations of detectable tags and a total number of detectable tags that is less than the total number of constituents to be encoded, to encode the complex mixture of assay constituents.

2. The method of claim 1, further comprising:
(a) determining the total number of constituents to be encoded;
(b) determining the number of detectable tags in each combination, wherein the number of detectable tags in each combination is more than one and less than or equal to the number of prime numbers in the number of constituents to be encoded; and
(c) determining the total number of detectable tags, wherein the total number of detectable tags equals a sum of a set of factors of the total number of constituents, wherein the number of factors equals the number of detectable tags in each combination.

3. The method of claim 2, wherein the total number of detectable tags is minimized by selecting factors of the total number of constituents that are equal or approximate.

4. The method of claim 1, wherein the constituents are selected from the group consisting of proteins, peptides, amino acids, small molecules, nucleotides, fatty acids, sugars, cofactors, receptors, receptor ligands, protein domains, oligonucleotides, transcription factors, nucleic acids, and small compounds.

5. The method of claim 1, wherein the detectable tags are directly or indirectly coupled to the constituents.

6. The method of claim 1, wherein the detectable tags are selected from the group consisting of radiolabels, dyes, fluorescent labels, and combinations thereof.

7. The method of claim 5, wherein the detectable tags are contained in or coupled to microspheres.

8. The method of claim 7, wherein the microspheres are coupled to a means of specifically binding the constituents.

9. The method of claim 8, wherein the coupled means is a nucleic acid complementary to a nucleic acid in or bound to the constituent to be bound.

10. A method of performing a multiplexed assay using complex mixtures of assay constituents encoded according to the method of claim 2, comprising:
(a) Performing an assay to produce assay products using an array of the complex mixtures, wherein each constituent in a single complex mixture is detectably tagged with a unique combination of detectable tags;
(b) Detecting which complex mixtures of assay products in the array have a positive response; and
(c) Decoding the constituents in the complex mixtures having the positive response to determine which specific constituent or constituents are positive.

11. The method of claim 10, wherein the assay is selected from the group consisting of a chemical assay, protein assay, pharmacologic assay, antibody binding assay, hybrid assay, display assay, and genomic assay readout assay.

12. The method of claim 10, wherein the constituents are selected from the group consisting of nucleic acids, proteins, peptides, amino acids, small molecules, nucleotides, fatty acids, sugars, cofactors, receptors, receptor ligands, protein domains, oligonucleotides, transcription factors, and small compounds.

13. The method of claim 10, wherein the detectable tags are directly or indirectly coupled to the constituents.

14. The method of claim 10, wherein the detectable tags are selected from the group consisting of radiolabels, dyes, fluorescent labels, and combinations thereof.

15. The method of claim 10, wherein the detectable tags are contained in or coupled to microspheres.

16. The method of claim 15, wherein the microspheres are coupled to a means of specifically binding the constituents.

17. The method of claim 16, wherein the coupled means is a nucleic acid complementary to a nucleic acid in or bound to the constituent to be bound to the microspheres.

18. The method of claim 10, wherein the decoding is performed by detecting and distinguishing with a detection device the detectable tags in each complex mixture of constituents.

19. A kit for performing a multiplexed assay using complex mixtures of encoded assay constituents, comprising a means of detectably tagging assay constituents encoded according to method of claim 2 and an arraying means for a plurality of complex mixtures.


SEQUENCE LISTING (29 sequences)

SEQ ID NO: 1 - DNA, 20 bp, Artificial Sequence
  Description of Artificial Sequence/note = synthetic construct
caggccaagtaacttcttcg

SEQ ID NO: 2 - DNA, 18 bp, Artificial Sequence
  Description of Artificial Sequence/note = synthetic construct
tgggcgacttctccaaac

SEQ ID NO: 3 - DNA, 18 bp, Artificial Sequence
  Description of Artificial Sequence/note = synthetic construct
cttgcagattcggcagtt

SEQ ID NO: 4 - DNA, 65 bp, Artificial Sequence
  Description of Artificial Sequence/note = synthetic construct
tgggcgacttctccaaacgatgatcgacgagacactctcgccactgtgacggaagatcacttcgc

SEQ ID NO: 5 - DNA, 65 bp, Artificial Sequence
  Description of Artificial Sequence/note = synthetic construct
tgggcgacttctccaaaccggtcgacgagctgccgcgcaagatctgtgacggaagatcacttcgc

SEQ ID NO: 6 - DNA, 65 bp, Artificial Sequence
  Description of Artificial Sequence/note = synthetic construct
tgggcgacttctccaaacgacattcgcgatcgccgcccgctttctgtgacggaagatcacttcgc

SEQ ID NO: 7 - DNA, 65 bp, Artificial Sequence
  Description of Artificial Sequence/note = synthetic construct
tgggcgacttctccaaaccggtatcgcgaccgcatcccaatctctgtgacggaagatcacttcgc

SEQ ID NO: 8 - DNA, 65 bp, Artificial Sequence
  Description of Artificial Sequence/note = synthetic construct
tgggcgacttctccaaacgctcgaagaggcgctacagatcctcctgtgacggaagatcacttcgc

SEQ ID NO: 9 - DNA, 65 bp, Artificial Sequence
  Description of Artificial Sequence/note = synthetic construct
tgggcgacttctccaaaccaccgccagctcggcttcgagttcgctgtgacggaagatcacttcgc

SEQ ID NO: 10 - DNA, 65 bp, Artificial Sequence
  Description of Artificial Sequence/note = synthetic construct
tgggcgacttctccaaacgtaaatctccagcggaagggtacggctgtgacggaagatcacttcgc

SEQ ID NO: 11 - DNA, 65 bp, Artificial Sequence
  Description of Artificial Sequence/note = synthetic construct
tgggcgacttctccaaaccttttcccgtccgtcatcgctcaagctgtgacggaagatcacttcgc

SEQ ID NO: 12 - DNA, 65 bp, Artificial Sequence
  Description of Artificial Sequence/note = synthetic construct
tgggcgacttctccaaacggctgggtctacagatccccaacttctgtgacggaagatcacttcgc

SEQ ID NO: 13 - DNA, 65 bp, Artificial Sequence
  Description of Artificial Sequence/note = synthetic construct
tgggcgacttctccaaacgaacctttcgcttcaccggccgatcctgtgacggaagatcacttcgc

SEQ ID NO: 14 - DNA, 65 bp, Artificial Sequence
  Description of Artificial Sequence/note = synthetic construct
tgggcgacttctccaaactttcggcacgcgcgggatcaccatcctgtgacggaagatcacttcgc

SEQ ID NO: 15 - DNA, 65 bp, Artificial Sequence
  Description of Artificial Sequence/note = synthetic construct
tgggcgacttctccaaacctcggtggtgctgacggtgcaatccctgtgacggaagatcacttcgc

SEQ ID NO: 16 - DNA, 65 bp, Artificial Sequence
  Description of Artificial Sequence/note = synthetic construct
cttgcagattcggcagtttcaacgtgccagcgccgtcctgggactccacggggagagcctgagca

SEQ ID NO: 17 - DNA, 65 bp, Artificial Sequence
  Description of Artificial Sequence/note = synthetic construct
cttgcagattcggcagttgcgaaggaactcgacgtggacgccgctccacggggagagcctgagca

SEQ ID NO: 18 - DNA, 65 bp, Artificial Sequence
  Description of Artificial Sequence/note = synthetic construct
cttgcagattcggcagttcggggataccgatctcgggcgcacactccacggggagagcctgagca

SEQ ID NO: 19 - DNA, 65 bp, Artificial Sequence
  Description of Artificial Sequence/note = synthetic construct
cttgcagattcggcagttggagcttacgccatcacgatgcgatctccacggggagagcctgagca

SEQ ID NO: 20 - DNA, 65 bp, Artificial Sequence
  Description of Artificial Sequence/note = synthetic construct
cttgcagattcggcagttcgtggcggtgcggagtttccccgaactccacggggagagcctgagca

SEQ ID NO: 21 - DNA, 65 bp, Artificial Sequence
  Description of Artificial Sequence/note = synthetic construct
cttgcagattcggcagttcgatccaacgcactggccaaacctactccacggggagagcctgagca

SEQ ID NO: 22 - DNA, 65 bp, Artificial Sequence
  Description of Artificial Sequence/note = synthetic construct
cttgcagattcggcagttctgaatcctccaaccgggttgtcgactccacggggagagcctgagca

SEQ ID NO: 23 - DNA, 65 bp, Artificial Sequence
  Description of Artificial Sequence/note = synthetic construct
cttgcagattcggcagttttcggcgctggcgtaaagcttttggctccacggggagagcctgagca

SEQ ID NO: 24 - DNA, 21 bp, Artificial Sequence
  Description of Artificial Sequence/note = synthetic construct
tccacggggagagcctgagca

SEQ ID NO: 25 - DNA, 22 bp, Artificial Sequence
  Description of Artificial Sequence/note = synthetic construct
ctgtgacggaagatcacttcgc

SEQ ID NO: 26 - DNA, 23 bp, Artificial Sequence
  Description of Artificial Sequence/note = synthetic construct
gcaacggcgactggctggaattc

SEQ ID NO: 27 - DNA, 23 bp, Artificial Sequence
  Description of Artificial Sequence/note = synthetic construct
gcttggctgcaggtcgactcgag

SEQ ID NO: 28 - DNA, 39 bp, Artificial Sequence
  Description of Artificial Sequence/note = synthetic construct
gccgaagcttgcggttggggtattcgcaacggcgactgg

SEQ ID NO: 29 - DNA, 40 bp, Artificial Sequence
  Description of Artificial Sequence/note = synthetic construct
atacgcatgcaattcgcccggaattagcttggctgcaggt